SEQUENCE LISTING (7 sequences)

SEQ ID NO: 1 - DNA, 52 bp, Homo sapiens
  misc_feature (34)..(34): n is g or a
ttttgatttcttcaccactatttgggatataacntgggttaacacagacata

SEQ ID NO: 2 - DNA, 49 bp, Homo sapiens
  misc_feature (25)..(25): n is g or a
cggacctcttgaggtcaggagttcnagaccagcctggccaacatggtga

SEQ ID NO: 3 - DNA, 46 bp, Homo sapiens
  misc_feature (21)..(21): n is g or t
aacagtctgtcagcaaagccngcagtacactgagaaagctcctatt

SEQ ID NO: 4 - DNA, 50 bp, Homo sapiens
  misc_feature (22)..(22): n is c or t
ccagaagttgaagtgtaggaanatctggggtcaaagcaaaaaaagacttt

SEQ ID NO: 5 - DNA, 56 bp, Homo sapiens
  misc_feature (25)..(25): n is c or t
tgggagtattttactgtgctaaaanacatttagcatgggctgttatatcttatgac

SEQ ID NO: 6 - DNA, 52 bp, Homo sapiens
  misc_feature (22)..(22): n is c or t
gataaaaaggaactgtttaaantgatagtaaagaaaagccttaaatttttgg

SEQ ID NO: 7 - DNA, 54 bp, Homo sapiens
  misc_feature (23)..(23): n is c or g
aataaaaacagaataaaacaatnagaaaattttcacctttatttaattagcaga

(19) United States
(12) Patent Application Publication
(10) Pub. No.: US 2006/0057626 A1
(43) Pub. Date: Mar. 16, 2006

(54) Assessment of CTLA-4 polymorphisms in CTLA-4 blockade therapy

(76) Inventors: Geoffrey M. Nichol, Short Hills, NJ (US); Michael Yellin, Upper Montclair, NJ (US); Steven Fischkoff, Short Hills, NJ (US); Jeffrey Weber, Los Angeles, CA (US)

Correspondence Address: DARBY & DARBY P.C., P. O. BOX 5257, NEW YORK, NY, 10150-5257, US

(21) Appl. No.: 11/218,284

(22) Filed: Aug. 31, 2005

Related U.S. Application Data
(60) Provisional application No. 60/607,225, filed on Sep. 3, 2004.
(60) Provisional application No. 60/611,831, filed on Sep. 20, 2004.

(51) Int. Cl. C12Q 1/68

(52) U.S. Cl. 435/6

(57) ABSTRACT

The invention provides methods for predicting responsiveness of a subject to therapy with a CTLA-4 blocking agent. The methods involve assaying for at least one CTLA-4 polymorphism in the subject and predicting responsiveness of the subject to therapy with a CTLA-4 blocking agent based on presence or absence of a CTLA-4 polymorphic allele in the subject. The methods can further comprise selecting a treatment regimen with a CTLA-4 blocking agent in a subject based upon presence or absence of a CTLA-4 polymorphic allele in the subject. The methods can further comprise administering a CTLA-4 blocking agent to the subject according to the selected treatment regimen. Kits comprising a CTLA-4 blocking agent and means for assaying one or more CTLA-4 polymorphisms, optionally including a vaccine, are also provided.

This application claims priority to U.S. Provisional Application No. 60/607,225, filed Sep. 3, 2004, and U.S. Provisional Application No. 60/611,831, filed Sep. 20, 2004. The contents of these applications are hereby incorporated by reference in their entirety.

### BACKGROUND OF THE INVENTION

CTLA-4 is a T cell surface molecule that was originally identified by differential screening of a murine cytolytic T cell cDNA library (Brunet et al. (1987) Nature 328:267-270). CTLA-4 is a member of the immunoglobulin (Ig) superfamily and comprises a single extracellular Ig domain. The human counterpart to the murine CTLA-4 cDNA has been identified (Dariavach et al. (1988) Eur. J. Immunol. 18:1901-1905) and the gene has been mapped to the same chromosomal region (2q33-34) as the T cell surface molecule CD28 (Lafage-Pochitaloff et al. (1990), Immuno-genetics 31:198-201). CTLA-4 and CD28 exhibit homology and both have been shown to bind to the B cell surface molecules B7-1 and B7-2. Whereas CD28 has been demonstrated to be a stimulatory molecule for T cell activation, CTLA-4 has been demonstrated to have an opposing role as a dampener of T cell activation (Krummel et al. (1995) J. Exp. Med. 182:459-465; Krummel et al. (1996) Int'l Immunol. 8:519-523; Chambers et al. (1997) Immunity. 7:885-895).

For example, it has been reported that CTLA-4 deficient mice suffer from massive lymphoproliferation (Chambers et al., supra). It also has been reported that CTLA-4 blockade augments T cell responses in vitro (Walunas et al. (1994) Immunity. 1:405-413) and in vivo (Kearney et al. (1995) J. Immunol. 155:1032-1036), exacerbates antitumor immunity (Leach et al. (1996) Science. 271:1734-1736), and enhances an induced autoimmune disease (Luhder et al. (1998) J. Exp. Med. 187:427-432). U.S. Pat. No. 5,855,887, U.S. Pat. No. 5,811,097 and U.S. Pat. No. 6,051,227 describe methods of increasing the response of a mammalian T cell to antigenic stimulation using a CTLA-4 blocking agent, such as an anti-CTLA-4 antibody, for example to increase anti-tumor responses in a tumor bearing subject.

Non-human CTLA-4 antibodies have been used in the various studies discussed above. Furthermore, human antibodies against human CTLA-4 have been described (e.g., PCT Publication WO 01/14424 and PCT Publication WO 00/37504). Human anti-CTLA-4 antibodies have been used clinically in humans and have been shown to increase T cell responses, such as anti-tumor responses (e.g., U.S. application Publication No. 2004-0005318). Anti-CTLA-4 therapy has also been shown to lead to autoimmune reactions (e.g., PCT Publication WO 00/3221), indicating that CTLA-4 blockade can overcome tolerance against self antigens.

Monoclonal antibody therapy has been used successfully in the treatment of cancer and other disorders and numerous monoclonal antibodies (chimeric, humanized or fully human) have been approved by the FDA for use in humans. While such therapy has demonstrated success, not all subjects treated respond, or respond as well as desired, to antibody therapy. Accordingly, methods for predicting and improving efficacy of antibody therapy are of great interest.

### SUMMARY OF THE INVENTION

This invention provides methods for predicting and improving the efficacy of therapy with CTLA-4 blocking agents that involve assessing CTLA-4 polymorphisms in the subject to be treated. The invention is based, at least in part, on the observation that the presence of certain CTLA-4 polymorphic alleles in subjects is associated with increased or decreased responsiveness to therapy with a CTLA-4 blocking agent. In particular, CTLA-4 polymorphic alleles that are associated with increased susceptibility to autoimmune disorders have been found to correlate with increased responsiveness to CTLA-4 blocking agent therapy, as evidenced by increased autoimmune reactions and decreased tumor progression in the subjects. In contrast, CTLA-4 polymorphic alleles that are associated with decreased susceptibility to autoimmune disorders (i.e., polymorphic alleles that are protective for susceptibility to autoimmune disorders) have been found to correlate with decreased responsiveness to CTLA-4 blocking agent therapy, as evidenced by decreased autoimmune reactions and increased tumor progression in the subjects. Accordingly, CTLA-4 polymorphisms can be assessed in subjects who are to undergo CTLA-4 blocking agent therapy to predict responsiveness of the subject to therapy and/or to aid in the selection of an appropriate treatment regimen.

In one aspect, the invention pertains to a method for predicting responsiveness of a subject to therapy with a CTLA-4 blocking agent, comprising:

assaying for at least one CTLA-4 polymorphism in the subject; and

predicting responsiveness of the subject to therapy with a CTLA-4 blocking agent based on presence or absence of a CTLA-4 polymorphic allele in the subject.

The method can further comprise selecting a treatment regimen with a CTLA-4 blocking agent based upon presence or absence of the CTLA-4 polymorphic allele in the subject. Accordingly, in another aspect, the invention pertains to a method for selecting a treatment regimen for therapy with a CTLA-blocking agent in a subject, comprising:

assaying for at least one CTLA-4 polymorphism in the subject; and

selecting a treatment regimen with a CTLA-4 blocking agent based upon presence or absence of a CTLA-4 polymorphic allele in the subject.

The method can further comprise administering the CTLA-4 blocking agent to the subject, for example to increase responsiveness to antigenic stimulation in the subject (e.g., to increase anti-tumor immunity in a tumor-bearing subject). Accordingly, in another aspect, the invention pertains to a method for increasing responsiveness to antigenic stimulation in a subject, comprising

assaying for at least one CTLA-4 polymorphism in the subject;

selecting a treatment regimen with a CTLA-4 blocking agent based upon presence or absence of a CTLA-4 polymorphic allele in the subject; and

administering the CTLA-4 blocking agent to the subject according to the treatment regimen such that responsiveness to antigenic stimulation is increased in the subject.

In the methods of the invention, the CTLA-4 polymorphism that is assayed can be, for example, a single nucleotide polymorphism (SNP) associated with susceptibility to autoimmune disease. A preferred SNP to be assayed is a JO30 G/A polymorphism. Other preferred SNPs to be assayed are a CT60 G/A polymorphism, a JO31 G/T polymorphism, a JO27_{—}1 T/C polymorphism, a CTAF343 T/C polymorphism, an rs1863800 C/T polymorphism and/or a MH30 G/C polymorphism.

In other embodiments, the CTLA-4 polymorphism to be assayed is a polymorphism associated with susceptibility to autoimmune disease selected from the group consisting of a CTLA-4 exon 1 position 49 A/G polymorphism, a CTLA-4 promoter position −318 C/T polymorphism, a CTLA-4 intron 1 position 1822 C/T polymorphism and a CTLA-4 exon 3 dinucleotide (AT)n repeat polymorphism.

In the methods of the invention for predicting responsiveness to therapy with a CTLA-4 blocking agent, when a CTLA-4 polymorphic allele associated with increased susceptibility to autoimmune disease is present in the subject, the subject is predicted to have increased responsiveness to therapy with a CTLA-4 blocking agent as compared to a subject not carrying the allele. Increased responsiveness to therapy with a CTLA-4 blocking agent can include at least one response selected from the group consisting of: increased T cell responsiveness to antigenic stimulation, increased anti-tumor activity, increased autoimmune breakthrough events, increased clinically adverse events and increased serological adverse events.

In the methods of the invention for predicting responsiveness to therapy with a CTLA-4 blocking agent, when a CTLA-4 polymorphic allele associated with decreased susceptibility to autoimmune disease is present in the subject, the subject is predicted to have decreased responsiveness to therapy with a CTLA-4 blocking agent as compared to a subject not carrying the allele. Decreased responsiveness to therapy with a CTLA-4 blocking agent can include at least one response selected from the group consisting of: decreased T cell responsiveness to antigenic stimulation, decreased anti-tumor activity, decreased autoimmune breakthrough events, decreased clinically adverse events and decreased serological adverse events.

In a preferred method for selecting a treatment regimen for therapy with a CTLA-4 blocking agent, the subject expresses two G alleles (G/G genotype) at a JO30 G/A polymorphism and the treatment regimen that is selected is a reduced treatment regimen as compared to a standard treatment regimen. In alternative embodiments, the subject expresses a genotype selected from the group consisting of two G alleles (G/G genotype) at a CT60 G/A polymorphism, two G alleles (G/G genotype) at a JO31 G/T polymorphism, two T alleles (T/T genotype) at a JO27_{—}1 T/C polymorphism, two T alleles (T/T genotype) at a CTAF343 T/C polymorphism, two C alleles (C/C genotype) at a rs1863800 C/T polymorphism and two G alleles (G/G genotype) at a MH30 G/C polymorphism and the treatment regimen that is selected is a reduced treatment regimen as compared to a standard treatment regimen. Examples of reduced treatment regimens include use of a lower dose of CTLA-4 blocking agent, less frequent administration of the CTLA-4 blocking agent or shorter treatment duration with the CTLA-4 blocking agent as compared to a standard treatment regimen.

In another preferred method for selecting a treatment regimen for therapy with a CTLA-4 blocking agent, the subject expresses two A alleles (A/A genotype) at a JO30 G/A polymorphism and the treatment regimen that is selected is an increased treatment regimen as compared to a standard treatment regimen. In alternative embodiments, the subject expresses a genotype selected from the group consisting of two A alleles (A/A genotype) at a CT60 G/A polymorphism, two T alleles (T/T genotype) at a JO31 G/T polymorphism, two C alleles (C/C genotype) at a JO27_{—}1 T/C polymorphism, two C alleles (C/C genotype) at a CTAF343 T/C polymorphism, two T alleles (T/T genotype) at a rs1863800 C/T polymorphism and two C alleles (C/C genotype) at a MH30 G/C polymorphism, and the treatment regimen that is selected is an increased treatment regimen as compared to a standard treatment regimen. Examples of increased treatment regimens include use of a higher dose of CTLA-4 blocking agent, more frequent administration of the CTLA-4 blocking agent or longer treatment duration with the CTLA-4 blocking agent as compared to a standard treatment regimen.

In the methods of the invention, CTLA-4 polymorphism can be assayed by any suitable technique known in the art, for example by polymerase chain reaction-restriction fragment length polymorphism (PCR-RFLP) analysis.

A preferred CTLA-4 blocking agent is an anti-CTLA-4 monoclonal antibody, such as a chimeric, humanized or human anti-CTLA-4 monoclonal antibody. Other examples of CTLA-4 blocking agents include aptamers that bind to CTLA-4, antisense agents that reduce expression of CTLA-4 and soluble peptide or protein ligands that bind to CTLA-4.

A preferred subject to be treated according to the methods of the invention is a human subject.

Another aspect of the invention pertains to kits that comprise:
a CTLA-4 blocking agent; and
means for assaying one or more CTLA-4 polymorphisms.

Preferred blocking agents are anti-CTLA-4 monoclonal antibodies, such as a chimeric, humanized or human anti-CTLA-4 monoclonal antibodies. Preferably, the means for assaying one or more CTLA-4 polymorphisms includes one or more polynucleotides specific for the polymorphisms.

In one embodiment, the kit can further comprise a vaccine, such as a tumor antigen or tumor cells transduced to secrete GM-CSF. Examples of tumor antigens include a gp100 peptide, prostate specific membrane antigen (PSMA) or a composition that comprises: 1) gp100 peptide, 2) a MART-I peptide and 3) a tyrosinase peptide.

### BRIEF DESCRIPTION OF THE DRAWINGS

FIG. 1 is a bar graph summarizing the incidence of clinical toxicity and disease progression, following treatment with MDX-010 and peptide vaccine, in subjects carrying the JO30 A/A polymorphism, the JO30 A/G polymorphism or the JO30 G/G polymorphism.

### DETAILED DESCRIPTION OF THE INVENTION

This invention provides methods for predicting responsiveness of a subject to therapy with a CTLA-4 blocking agents, and methods for selecting a treatment regimen with a CTLA-4 blocking agent, based on expression of CTLA-4 polymorphic alleles in the subject to be treated. The invention is based, at least in part, on the observation that the presence of certain CTLA-4 polymorphic alleles in subjects is associated with increased or decreased responsiveness to therapy with a CTLA-4 blocking agent (see Example 1). In particular, CTLA-4 polymorphic alleles that are associated with increased susceptibility to autoimmune disorders have been found to correlate with increased responsiveness to CTLA-4 blocking agent therapy, as evidenced by increased autoimmune reactions and decreased disease progression in the subjects. In contrast, CTLA-4 polymorphic alleles that are associated with decreased susceptibility to autoimmune disorders (i.e., polymorphic alleles that are protective for susceptibility to autoimmune disorders) have been found to correlate with decreased responsiveness to CTLA-4 blocking agent therapy, as evidenced by decreased autoimmune reactions and increased disease progression in the subjects. Accordingly, CTLA-4 polymorphisms can be assessed in subjects who are to undergo CTLA-4 blocking agent therapy to predict responsiveness of the subject to therapy and/or to aid in the selection of an appropriate treatment regimen.

In order that the present invention may be more readily understood, certain terms are first defined. Additional definitions are set forth throughout the detailed description.

The terms “cytotoxic T lymphocyte-associated antigen-4,” “CTLA-4,” “CTLA4,” “CTLA-4 antigen” and “CD152” are used interchangeably and refer to the same protein. The complete cDNA sequence encoding the human CTLA-4 protein has the Genbank accession number L15006. The complete cDNA sequence encoding the mouse CTLA-4 protein has the Genbank accession number NM_{—}009843.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

The term “CTLA-4 blockade” is intended to refer to disruption or inhibition of the immunoinhibitory effect of CTLA-4 such that immune responses are upregulated. The term “CTLA-4 blocking agent” is intended to refer to an agent capable of disrupting or inhibiting the immunoinhibitory effect of CTLA-4 such that immune responses are upregulated.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., IP-10). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to refer to antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell. The term “human monoclonal antibody”, as used herein, also includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, the term “subject” includes humans, and non-human animals amenable to CTLA-4 blocking agent therapy, e.g., preferably mammals, such as non-human primates, sheep, dogs, cats, horses and cows.

Various aspects of the invention are described in further detail in the following subsections.

CTLA-4 Polymorphisms

Susceptibility to autoimmune disorders such as Graves' disease, autoimmune hypothyroidism (AIH) and type 1 diabetes (T1D) has been mapped to a non-coding 6.1 kb 3′ region of CTLA-4 (Ueda, H. et al. (2003) Nature 423:506-511, the entire contents of which, including Supplementary Information A and B, are hereby specifically incorporated by reference). Seven single nucleotide polymorphisms (SNPs) were identified as being most closely associated with susceptibility to autoimmune disease. These markers are the CT60, JO30, JO31, JO27_{—}1, CTAF343, rs1863800 and MH30 polymorphisms and are described in further detail in Example 2.

For the CT60 G/A polymorphism, the G allele has been correlated with susceptibility to autoimmunity, the A allele is protective (i.e., the “protective” allele is not associated with an increased risk of autoimmunity).

For the JO30 G/A polymorphism, the G allele has been correlated with susceptibility to autoimmunity, the A allele is protective.

For the JO31 G/T polymorphism, the G allele has been correlated with susceptibility to autoimmunity, the T allele is protective.

For the JO27_{—}1 T/C polymorphism, the T allele has been correlated with susceptibility to autoimmunity, the C allele is protective.

For the CTAF343 T/C polymorphism, the T allele has been correlated with susceptibility to autoimmunity, the C allele is protective.

For the rs1863800 C/T polymorphism, the C allele has been correlated with susceptibility to autoimmunity, the T allele is protective.

For the MH30 G/C polymorphism, the G allele has been correlated with susceptibility to autoimmunity, the C allele is protective.

Another preferred CTLA-4 polymorphism for use in the invention is the JO33 G/A polymorphism, in which the G allele has been correlated with susceptibility to autoimmunity and the A allele is protective (see Example 1).

Other CTLA-4 polymorphisms have been reported to be associated with susceptibility to various autoimmune disorders. Examples of these include:

1) A CTLA-4 exon 1 position 49 A/G polymorphism, wherein the G allele has been correlated with susceptibility to autoimmunity and the A allele is “protective” (see e.g., Donner, H. et al. (1997) J. Clin. Endocrinol. Metab. 82:4130-4132; Kouki, T. et al. (2000) J. Immunol. 165:6606-6611; Rau, H. et al. (2001) J. Clin. Endocrinol. Metab. 86:653-655);

2) A CTLA-4 promoter position -318 C/T polymorphism, wherein the C allele has been correlated with susceptibility to autoimmunity and the T allele is “protective” (see e.g., Park, Y. J. et al. (2000) Thyroid 10:453-459);

3) A CTLA-4 intron 1 position 1822 C/T polymorphism, wherein the T allele has been correlated with susceptibility to autoimmunity and the C allele is “protective” (see e.g., Vaidya, B. et al. (2003) Clin. Endocrinol. 58:732-735); and

4) A CTLA-4 exon 3 dinucleotide (AT)n repeat polymorphism, wherein the 106 base pair allele has been correlated with susceptibility to autoimmunity (see e.g., Kotsa, K. et al. (1997) Clin. Endocrinol. 46:551-554; Kemp, E. H. et al. (1998) Clin. Endocrinol. 49:609-613).

Although the Ueda et al. study described above reports that none of these four polymorphisms are the causal variant of increased autoimmune susceptibility, and thus any correlation with these markers is due to linkage disequilibrium, nevertheless these markers may be suitable for use in the methods of the invention, although the JO30, CT60, JO31, JO27_{—}1, CTAF343, rs1863800 and MH30 polymorphisms are more preferred for use due to their closer correlation with autoimmune susceptibility. A particularly preferred polymorphism to be assayed is the J030 polymorphism, as described in Example 1.

Although not intending to be limited by mechanism, Ueda et al. report that mRNA levels of a soluble splice-variant form of CTLA-4 (sCTLA-4) from a disease-protective haplotype are higher than mRNA levels of sCTLA-4 from a disease-susceptible haplotype (Ueda et al., supra). Accordingly, in addition to the polymorphisms discussed herein, other CTLA-4 polymorphisms that may suitable for use in the invention include polymorphisms associated with decreased mRNA levels of sCTLA-4, regardless of whether the polymorphism has been demonstrated to be correlated with autoimmune susceptibility.

Assaying CTLA-4 Polymorphisms

CTLA-4 polymorphisms can be assayed in the methods of the invention using any suitable technique known in the art for evaluating genetic polymorphisms. For example, a standard method for assessing polymorphisms is by polymerase chain reaction-restriction fragment length polymorphism (PCR-RFLP). For typing large numbers of single nucleotide polymorphisms (SNPs) rapidly, efficiently and accurately, the Invader® technology can be used (described further in Mein, C. et al. (2000) Genome Research 10:330-343, the contents of which is expressly incorporated herein by reference). Alternatively, SNPs can be evaluated using an amplification refractory mutation system-polymerase chain reaction (ARMS-PCR) technology such as described in Perrey, C. et al. (1999) Transplant Immunol. 7:127-128. Dinucleotide repeat polymorphisms, such as the (AT)n repeat in exon 3 of CTLA-4, can be evaluated by, for example, the method described in Polymeropoulos, M. H. et al. (1991) Nucl. Acids Res. 19:4018.

Predicting Responsiveness to CTLA-4 Blockade Therapy

As discussed further in Example 1, a correlation has been demonstrated between the presence of a CTLA-4 polymorphic allele associated with susceptibility to autoimmunity and increased responsiveness to treatment with a CTLA-4 blocking agent. According, the invention provides a method for predicting responsiveness of a subject to therapy with a CTLA-4 blocking agent, comprising:

assaying for at least one CTLA-4 polymorphism in the subject; and

predicting responsiveness of the subject to therapy with a CTLA-4 blocking agent based on presence or absence of a CTLA-4 polymorphic allele in the subject.

In the prediction methods of the invention, when a CTLA-4 polymorphic allele associated with increased susceptibility to autoimmune disease is present in the subject, the subject is predicted to have increased responsiveness to therapy with a CTLA-4 blocking agent as compared to a subject not carrying the allele. Increased responsiveness to therapy with a CTLA-4 blocking agent can include at least one response selected from the group consisting of: increased T cell responsiveness to antigenic stimulation, increased anti-tumor activity, increased autoimmune breakthrough events, increased clinically adverse events and increased serological adverse events.

In the prediction methods of the invention, when a CTLA-4 polymorphic allele associated with decreased susceptibility to autoimmune disease (e.g., a “protective” allele) is present in the subject, the subject is predicted to have decreased responsiveness to therapy with a CTLA-4 blocking agent as compared to a subject not carrying the allele. Decreased responsiveness to therapy with a CTLA-4 blocking agent can include at least one response selected from the group consisting of: decreased T cell responsiveness to antigenic stimulation, decreased anti-tumor activity, decreased autoimmune breakthrough events, decreased clinically adverse events and decreased serological adverse events.

Selection of CTLA-4 Blocking Agent Treatment Regimens

Given the observation that the presence or absence of particular CTLA-4 polymorphic alleles in a subject influences the responsiveness of the subject to therapy with a CTLA-4 blocking agent, one can select an appropriate treatment regimen for the subject based on the presence or absence of particular CTLA-4 polymorphic alleles. Accordingly, the invention provides a method for selecting a treatment regimen for therapy with a CTLA-blocking agent in a subject, comprising:

assaying for at least one CTLA-4 polymorphism in the subject; and

selecting a treatment regimen with a CTLA-4 blocking agent based upon presence or absence of a CTLA-4 polymorphic allele in the subject.

The treatment regimen that is selected can be, for example, a reduced treatment regimen as compared to a standard treatment regimen or an increased treatment regimen as compared to a standard treatment regimen. A “standard treatment regimen” is a regimen that would be selected for the subject if the subject were not screened for a CTLA-4 polymorphism(s) before treatment, and includes art-accepted (e.g., FDA approved) treatment regimens that are not restricted for use in particular patient populations that carry particular CTLA-4 polymorphic alleles.

Since the presence of a CTLA-4 polymorphic allele associated with autoimmune disease susceptibility has been shown to correlate with increased responsiveness to CTLA-4 blocking agent therapy, a reduced treatment regimen can be selected for subjects that carry an autoimmunity-associated CTLA-4 polymorphic allele. This reduced treatment regimen may be preferred for the subject in order to reduce the extent, severity or duration of autoimmune breakthrough events while still maintaining efficacy in stimulation of desired immune responses (e.g., anti-tumor immunity). A reduced treatment regimen can comprise, for example, use of a lower dose of CTLA-4 blocking agent, less frequent administration of the CTLA-4 blocking agent or shorter treatment duration with the CTLA-4 blocking agent as compared to a standard treatment regimen.

For example, if a standard treatment regimen comprises use of 3 mg/kg of agent, a reduced treatment regimen may comprise use of less than 3 mg/kg, such as 2 mg/kg, 1 mg/kg, 0.5 mg/kg or 0.1 mg/kg of agent. Alternatively, if a standard treatment regimen comprises uses 5 mg/kg of agent, a reduced treatment regimen may comprise use of less than 5 mg/kg, such as 4 mg/kg, 3 mg/kg, 2 mg/kg, 1 mg/kg, 0.5 mg/kg or 0.1 mg/kg of agent. Alternatively, if a standard treatment regimen comprises uses 1 mg/kg of agent, a reduced treatment regimen may comprise use of less than 1 mg/kg, such as 0.5 mg/kg or 0.1 mg/kg of agent.

Also for example, if a standard treatment regimen comprises administration of agent every four weeks, a reduced treatment regimen may comprise administration less frequently, such as every five weeks, every six weeks, every seven weeks, every eight weeks, every nine weeks, every ten weeks, every eleven weeks or every twelve weeks. Alternatively, if a standard treatment regimen comprises administration of agent every two weeks, a reduced treatment regimen may comprise administration less frequently, such as every three weeks, every four weeks, every five weeks, every six weeks, every seven weeks, every eight weeks, every nine weeks, every ten weeks, every eleven weeks or every twelve weeks.

Also for example, if a standard treatment regimen comprises treatment with the agent for a duration of 12 months, a reduced treatment regimen may comprise treatment for a shorter duration, such as for three months, six months or nine months. Alternatively, if a standard treatment regimen comprises treatment with the agent for a duration of 6 months, a reduced treatment regimen may comprise treatment for a shorter duration, such as for one month, two months, three months, four months or five months.

In a preferred method for selecting a treatment regimen for therapy with a CTLA-4 blocking agent, the subject expresses two G alleles (G/G genotype) at a JO30 G/A polymorphism and the treatment regimen that is selected is a reduced treatment regimen as compared to a standard treatment regimen. In alternative embodiments, the subject expresses a genotype selected from the group consisting of two G alleles (G/G genotype) at a CT60 G/A polymorphism, two G alleles (G/G genotype) at a JO31 G/T polymorphism, two T alleles (T/T genotype) at a JO27_{—}1 T/C polymorphism, two T alleles (T/T genotype) at a CTAF343 T/C polymorphism, two C alleles (C/C genotype) at a rs1863800 C/T polymorphism and two G alleles (G/G genotype) at a MH30 G/C polymorphism and the treatment regimen that is selected is a reduced treatment regimen as compared to a standard treatment regimen.

In an alternative embodiment, since the presence of a CTLA-4 polymorphic allele associated with reduced risk for autoimmunity has been shown to correlate with decreased responsiveness to CTLA-4 blocking agent therapy, an increased treatment regimen can be selected for subjects that carry an autoimmune-protective CTLA-4 polymorphic allele. This increased treatment regimen may be preferred or necessary in the subject in order to improve the efficacy of CTLA-4 blockade therapy and achieve the desired stimulation of immune responses (e.g., anti-tumor immunity), since these subjects may be more resistant to breaking of immune tolerance (as evidenced by decreased incidence of autoimmune breakthough events in these subjects). An increased treatment regimen can comprise, for example, use of a higher dose of CTLA-4 blocking agent, more frequent administration of the CTLA-4 blocking agent or longer treatment duration with the CTLA-4 blocking agent as compared to a standard treatment regimen.

For example, if a standard treatment regimen comprises use of 3 mg/kg of agent, an increased treatment regimen may comprise use of more than 3 mg/kg, such as 4 mg/kg, 5 mg/kg, 6 mg/kg, 7 mg/kg, 8 mg/kg, 9/mg/kg or 10 mg/kg of agent. Alternatively, if a standard treatment regimen comprises uses 1 mg/kg of agent, an increased treatment regimen may comprise use of more than 1 mg/kg, such as 2 mg/kg, 3 mg/kg, 4 mg/kg, 5 mg/kg, 6 mg/kg, 7 mg/kg, 8 mg/kg, 9/mg/kg or 10 mg/kg of agent.

Also for example, if a standard treatment regimen comprises administration of agent every four weeks, an increased treatment regimen may comprise administration more frequently, such as every three weeks, every two weeks or every week. Alternatively, if a standard treatment regimen comprises administration of agent every three months, an increased treatment regimen may comprise administration more frequently, such as every two months, every month, every four weeks, every three weeks, every two weeks or every week.

Also for example, if a standard treatment regimen comprises treatment with the agent for a duration of 9 months, an increased treatment regimen may comprise treatment for a longer duration, such as for ten months, eleven months, twelve months, fifteen months or eighteen months. Alternatively, if a standard treatment regimen comprises treatment with the agent for a duration of 6 months, an increased treatment regimen may comprise administration for a longer duration, such as for seven months, eight months, nine months, ten months, eleven months, twelve months, fifteen months or eighteen months.

In another preferred method for selecting a treatment regimen for therapy with a CTLA-4 blocking agent, the subject expresses two A alleles (A/A genotype) at a JO30 G/A polymorphism and the treatment regimen that is selected is an increased treatment regimen as compared to a standard treatment regimen. In alternative embodiments, the subject expresses a genotype selected from the group consisting of two A alleles (A/A genotype) at a CT60 G/A polymorphism, two T alleles (T/T genotype) at a JO31 G/T polymorphism, two C alleles (C/C genotype) at a JO27_{—}1 T/C polymorphism, two C alleles (C/C genotype) at a CTAF343 T/C polymorphism, two T alleles (T/T genotype) at a rs1863800 C/T polymorphism and two C alleles (C/C genotype) at a MH30 G/C polymorphism, and the treatment regimen that is selected is an increased treatment regimen as compared to a standard treatment regimen.

Administration of CTLA-4 Blocking Agents

Once an appropriate treatment regimen is selected for a subject based on presence or absence of a CTLA-4 polymorphic allele, a CTLA-4 blocking agent can be administered to the subject to increase responsiveness to antigenic stimulation in a subject. Accordingly, in another aspect, the invention pertains to a method for increasing responsiveness to antigenic stimulation in a subject, comprising

assaying for at least one CTLA-4 polymorphism in the subject;

selecting a treatment regimen with a CTLA-4 blocking agent based upon presence or absence of a CTLA-4 polymorphic allele in the subject; and

administering the CTLA-4 blocking agent to the subject according to the treatment regimen such that responsiveness to antigenic stimulation is increased in the subject.

The methods of the invention for increasing responsiveness to antigenic stimulation can be used in any clinical indication or setting in which an increased antigenic response is desired, for example to increase anti-tumor immunity in tumor bearing subject, to increase anti-bacterial immunity is a subject with a bacterial infection, to increase anti-viral activity in a subject with a viral infection or to increase the effectiveness of vaccination in a subject being vaccinated as a preventative measure for pathogenic infection.

Any CTLA-4 blocking agent that disrupts or inhibits the downregulatory effect of CTLA-4 on immune responsiveness can be used in the method. Preferred CTLA-4 blocking agents are anti-CTLA-4 monoclonal antibodies, such a human, humanized or chimeric monoclonal antibodies. Human anti-CTLA-4 monoclonal antibodies are known in the art. For example, the preparation and structural characterization of fully human antibodies that bind CTLA-4 are described in detail in, for example, PCT Publication WO 01/14424, PCT Publication WO 00/37504 and U.S. Pat. No. 6,682,736. A preferred anti-CTLA-4 human monoclonal antibody is MDX-010 (also known as 10D1), the structure of which is described in PCT Publication WO 01/14424. Non-human anti-CTLA-4 antibodies, such as murine antibodies, are also known in the art (see e.g., Linsely, P. S. et al. (1992) J. Exp. Med. 176:1595-1604). The variable regions of such non-human anti-CTLA-4 antibodies, and the CDR regions therein, can be used to prepare chimeric and humanized antibodies using techniques well established in the art.

Another type of CTLA-4 blocking agent is an RNA aptamer. Multivalent RNA aptamers that bind to CTLA-4 with high affinity, inhibit CTLA-4 function in vitro and enhance tumor immunity in vivo have been described (Santulli-Marotto, S. et al. (2003) Cancer Res. 63:7483-7489) and can be used in the treatment methods of the invention.

Other types of CTLA-4 blocking agents include antisense agents that reduce expression of CTLA-4, peptides derived from CTLA-4 ligands (such as B7-1 or B7-2 peptides) that bind to CTLA-4 and inhibit its activity and small molecule inhibitors that are selected for inhibiting the activity of CTLA-4.

For administration to a subject, a CTLA-4 blocking agent typically is formulated into a pharmaceutical compositions containing the CTLA-4 blocking agent and a pharmaceutically acceptable carrier. Therapeutic compositions typically must be sterile and stable under the conditions of manufacture and storage. Pharmaceutical compositions of the invention also can be administered in combination therapy, i.e., combined with other agents, such as other CTLA-4 blocking agents, other therapeutic agents (such as chemotherapeutic agents for the treatment of tumors) and vaccines that contain an antigen to which an increased immune response is desired.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

An agent of the present invention can be administered via one or more routes of administration using one or more of a variety of methods known in the art. As will be appreciated by the skilled artisan, the route and/or mode of administration will vary depending upon the desired results. A preferred route of administration, particularly for antibody agents, is by intravenous injection or infusion. Other preferred routes of administration include intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an agent of the invention can be administered via a non-parenteral route, such as a topical, epidermal or mucosal route of administration, for example, intranasally, orally, vaginally, rectally, sublingually or topically.

The CTLA-4 blocking agent also can be used in combination therapy in which the treatment regimen includes use of one or more additional agents other than the CTLA-4 blocking agent. A preferred additional agent for combination therapy is a vaccine that contains an antigen to which an increased immune response is desired. For example, for treatment of tumors, treatment with the CTLA-4 blocking agent can be combined with treatment with a tumor vaccine that contains tumor antigens. Non-limiting examples of tumor antigens include purified peptides from proteins that are preferentially expressed on tumor cells and tumor cells transduced to secrete GM-CSF. Non-limiting specific examples of tumor antigens include a gp100 peptide, prostate specific membrane antigen (PSMA) or a composition that comprises: 1) gp100 peptide, 2) a MART-1 peptide and 3) a tyrosinase peptide.

Other forms of combination therapy include administration of the CTLA-4 blocking agent with one or more chemotherapeutic drugs in the treatment of a tumor in a subject or administration of one or more antibiotic or anti-viral drugs in the treatment of a bacterial or viral infection in a subject.

Kits of the Invention

The invention also provides kits that can be used in practicing the methods of the invention. The kit can include:
a CTLA-4 blocking agent; and
means for assaying one or more CTLA-4 polymorphisms.

Preferred blocking agents are anti-CTLA-4 monoclonal antibodies, such as chimeric, humanized or human anti-CTLA-4 monoclonal antibodies. Other blocking agents include those described in the previous section.

Preferably, the means for assaying one or more CTLA-4 polymorphisms includes one or more polynucleotides specific for the polymorphisms. The means for assaying the polymorphism(s) can also include, for example, buffers or other reagents for use in an assay for evaluating the polymorphism(s) and printed instructions for performing the assay for evaluating the polymorphism(s)

In one embodiment, the kit can further comprise a vaccine. The vaccine can comprise an antigen to which immune responses are to be stimulated using CTLA-4 blockade therapy, such as a tumor antigen, a bacterial antigen or a viral antigen. Non-limiting examples of tumor antigens include purified peptides from proteins that are preferentially expressed on tumor cells and tumor cells transduced to secrete GM-CSF. Non-limiting specific examples of tumor antigens include a gp100 peptide, prostate specific membrane antigen (PSMA) or a composition that comprises: 1) gp100 peptide, 2) a MART-1 peptide and 3) a tyrosinase peptide.

The present invention is further illustrated by the following examples which should not be construed as further limiting. The contents of all references, patents and published patent applications cited throughout this application are expressly incorporated herein by reference.

### EXAMPLE 1

### Correlation of Increased Responsiveness to CTLA-4 Blocking Agent Therapy with Presence of Autoimmune Disease-Associated CTLA-4 Polymorphism

In this example, a clinical study was conducted in which human patients with advanced melanoma were treated with a CTLA-4 blocking agent (the anti-CTLA-4 human monoclonal antibody MDX-010) and a peptide vaccine containing three melanoma antigen peptides (gp100, tyrosinase, MART-1). Each of three cohorts received escalating doses of antibody with vaccine to primarily evaluate the toxicities and maximum tolerated dose (MTD) of antibody with vaccine. MDX-010 pharmacokinetics and immune responses were secondary endpoints.

Responsiveness to anti-CTLA-4 therapy was assessed and the patients were screened for the CTLA-4 JO30 G/A polymorphism, which is associated with susceptibility to autoimmune disease. The results demonstrate a correlation between the presence of the disease-associated G/G genotype and an increased incidence of clinical toxicity and decreased incidence of disease progression. In contrast, patients carrying the protective A allele (A/A genotype or A/G genotype) exhibited decreased incidence of clinical toxicity (with lowest toxicity seen with the A/A genotype) and increased incidence of disease progression (with greatest progression seen with the A/A genotype). The results indicate that presence of the G/G polymorphism associated with autoimmune disease correlates with increased responsiveness to CTLA-4 blocking agent therapy, as evidenced by increased autoimmune events and decreased tumor progression. In contrast, presence of the A/A polymorphism that is protective against autoimmune disease correlates with decreased responsiveness to CTLA-4 blocking agent therapy, as evidenced by decreased autoimmune events (indicating the blocking agent was less effective at breaking tolerance) and increased tumor progression.

The materials and methods used in the study, and the results obtained, are described in further detail below.

Materials and Methods

Patients

Patients had resected stage III or IV melanoma by the 2001 modified American Joint Commission on Cancer staging system and were rendered free of disease surgically. They had a magnetic resonance imaging or computed tomographic scan of the brain and computed tomographic imaging of the chest, abdomen, and pelvis performed within 4 weeks of therapy showing no evidence of disease. Eligibility criteria included age 18 or greater, creatinine of less than 2.0 mg/dl, bilirubin of less than 2.0 mg/dl, platelet count of 100,000/mcl or more, hemoglobin of 9 g/dl or more, and total WBC of 3000/mcl or greater. Human immunodeficiency virus, hepatitis C antibody and hepatitis B surface antigen titers were required to be negative. All patients were HLA-A*0201 positive by a DNA polymerase chain reaction (PCR) assay. Exclusion criteria included active autoimmune disease, steroid dependence and prior treatment with MDX-010 antibody or MART-1, gp100 and tyrosinase peptides.

Antibody Material

MDX-010 (anti-CTLA4 monoclonal antibody) is a fully human immunoglobulin (IgG₁) anti-CTLA4 monoclonal antibody. It was supplied at a concentration of 5 mg/ml in vials containing 5 or 10 ml and was stored at a temperature between 2° C. and 8° C. MDX-010 was drawn through a 0.22 μM filter and diluted in normal saline to a concentration of 2.5 mg/ml and was administered over a period of 90 minutes.

Peptide Vaccine Material

The tyrosinase₃₆₈₋₃₇₆ (370D) peptide [NSC# 699048], MART-1₂₆₋₃₅ (27L) peptide [NSC# 709401], and gp100₂₀₉₋₂₁₇ (210M) peptide, [NSC #683472] HLA-A*0201 restricted 9 or 10 amino acid epitope peptides were prepared to GMP grade and administered as previously described (Pullarkat et al. (2003) Clin. Cancer Res. 4:1301-1312). They were supplied by Ben Venue Laboratories, Inc. (Bedford, Ohio).

Montanide ISA-51 (also known as Incomplete Freund's Adjuvant or IFA), was manufactured by Seppic, Inc., (Franklin Lakes, N.J.) and supplied by CTEP/NCI in glass ampoules containing 3 ml of sterile adjuvant solution without preservative.

Treatment Regimen

MDX-010 was administered intravenously over 90 minutes every four weeks for 6 months and then every 12 weeks for 6 months. Antibody infusions were accompanied by three separate subcutaneous injection of 1 mg of each peptide emulsified in Incomplete Freund's Adjuvant (Montanide ISA 51) in one extremity. Patient cohorts received MDX-010 at 0.3, 1.0 or 3.0 mg/kg followed by the subcutaneous injection of tyrosinase₃₆₈₋₃₇₆ (370D), gp100₂₀₉₋₂₁₇ (210M) and MART-1₂₆₋₃₅ (27L) peptides.

A leukapheresis procedure with exchange of 5 to 7 liters to obtain peripheral blood mononuclear cells for immune analyses was performed immediately before and six months after the initial vaccination. Patients were followed until relapse.

Pheresis samples were processed to purify PBMCs and frozen at −168 degrees Celsius as previously described (Lee et al. (2001) J. Clin. Oncol. 19:3836-3847).

Trial Endpoints

The primary endpoints of the trial were a determination of the side effects and tolerability of MDX-010 treatment, and a determination of the maximum tolerated dose (MTD) of MDX-010 when given with a vaccine regimen. Secondary endpoints included the pharmacokinetics of MDX-010 and immunologic responses to the vaccine with MDX-010.

ELISPOT and Tetramer Assays

PBMC were thawed and cultured overnight then tested in an ELISPOT assay set up as previously described (Pullarkat et al., supra). Membrane plates (MAHA S45-10, Millipore, Bedford, Mass.) were prepared by adding primary anti-gamma interferon antibody (MabTech, Nacka, Sweden) and placed overnight at 4° C. in a refrigerator. The next day, plates were washed, and incubated for at least 1 hour at 37° C. with blocking buffer (AIM-V medium with 10% human AB serum). PBMC were added at 166,000, 83,000, and 41,500 cells per well in triplicate in a total volume of 100 ul. PHA at 10 ug/ml was added to six wells as a positive control, and AIM-V media as a negative control. Peptides were then added at 5 ug/ml to all other wells. Plates were then incubated in a 5% CO₂ incubator for 4 hours at 37° C. then washed. Secondary antibody (MabTech) at 1 mg/ml was then added. The plates were incubated overnight at 4° C., washed then blotted dry, and strepavidine/alkaline phosphatase (MabTech) with 1% BSA (Sigma) was added. BCIP/NBT (Kirkegaard & Perry, Gaithersburg, Md.) was added and plates incubated in the dark for development. The colorimetric reaction was then halted by washing with running water. After processing, ELISPOT plates were read on a KS Elispot reader (Carl Zeiss, Thornwood, N.Y.). Values were normalized to spots per 100,000 CD8 T cells. Negative controls included the HPV E7 86-93 A*0201 restricted peptide, for which geometric mean values were characteristically less than 10 spots per 10⁵. Assays were performed using both substituted gp100₂₀₉₋₂₁₇ (210M), MART-1₂₆₋₃₅ (27L) and tyrosinase₃₆₈₋₃₇₆ (370D), as well as wild-type gp100₂₀₉₋₂₁₇ and MART-1₂₇₋₃₅ peptides.

Tetramers containing the gp100₂₀₉₋₂₁₇ (210M), MART-1₂₆₋₃₅ (27L) and tyrosinase₃₆₈₋₃₇₆ (370D) peptides, for use in assays, were produced following the approach of Altman (Altman et al. (1998) Science 274:94-96). The tetramer assay technique has been previously published (Weber, J. et al. (2003) Cancer 97:186-200; Pullarkat et al., supra).

Each tetramer was validated by staining against a CTL line or clone specific for HLA-A2 in association with the peptide of interest. The limit of detection was 0.01% of CD8+ T cells as previously described (Pullarkat et al., supra).

Statistical differences in the pre- and post-vaccination values for gp100₂₀₉₋₂₁₇ (210M), MART-1₂₆₋₃₅ (27L) or tyrosinase₃₆₈₋₃₇₆ (370D) peptides for ELISPOT, and tetramer assays were examined. Positive responses were defined as a post-vaccine value greater than the pre-vaccine value of at least three times the standard deviation of the mean of the pre-vaccine value. A nonparametric test (Wilcoxon Rank Sum) test was used to compare differences between the cohorts.

Skin Tests

Skin tests were performed as previously described (Lee, P et al. (2001) J. Clin. Oncol. 19:3836-3847).

All patients had a complete skin examination prior to therapy and at each visit for vaccination to screen for vitiligo. Ocular toxicity was determined as previously described (Pullarkat et al., supra).

FACS Analysis

PBMC from patients were stained with FITC-labeled anti-CD8, anti-CCR9, anti-CLA, PE-conjugated peptide/HLA-A2.1 tetramers as well as CCR4 antibodies, and Cy5-labeled anti-CD4, 14, and 19 antibodies at 4 degrees Celsius for 30 minutes. Cells were washed and analyzed on a FACSCalibur (Becton Dickinson, San Jose, Calif.) as previously described (Pullarkat et al., supra).

Immunohistochemistry

Immunohistochemical staining of paraffin imbedded sections on glass slides for HMB-45 (gp100), MART-1 and tyrosinase was performed as previously described (Pullarkat et al., supra). Appropriate negative and positive control sections were included with each assay.

Archival formalin-fixed, paraffin embedded tissue was sectioned at 5 μm intervals and mounted on charged slides (ProbeOn+ Fisher Scientific, Pittsburgh, Pa.). Tissue sections were deparaffinized and rehydrated through graded alcohols. Tissue was then subjected to antigen-retrieval. Thereafter, the tissue was incubated with a blocking serum (usually 5% horse serum). The blocking serum was then aspirated and the primary monoclonal antibody was applied in the appropriate dilution. Anti-CD4 and anti-CD8 monoclonal antibodies (Becton Dickinson, Mountain View, Calif.) were used for immunohistochemical staining of paraffin imbedded sections on glass slides. Incubation with the primary antibody took place at room temperature or at 4° C., usually either 1 hour or overnight, depending on the system (optimized for each antibody). This was followed by a wash and then visualization with an avidin-biotin complex immunoperoxidase system (Vector Labs, Burlingame, Calif.) using 0.03% diamino-benzidine (DAB) as the chromagen and hematoxylin as the counterstain. In all cases, where appropriate, both external and internal controls were used to assess the quality of the IHC reaction.

Plasma Levels of MDX-010

Plasma samples were stored at −80° C. until analysis. A quantitative, functional ELISA was used to determine plasma levels of MDX-010. In this assay, plasma samples were incubated on a plate coated with a recombinant human CTLA4 fusion protein. The bound MDX-010 was detected with an anti-human IgG, F(ab)′2 alkaline phosphatase probe. A standard curve was generated and plasma levels were calculated from the linear portion of the standard curve.

Plasma Levels of Anti-MDX-010

Plasma samples for anti-MDX-010 antibody analysis were stored at −80° C. until analysis. A semi-quantitative ELISA was used to determine the level of plasma anti-MDX-010 IgG. Plates were coated with MDX-010 F(ab′)₂ and antibodies were detected with an anti-human IgG, Fc specific conjugated probe. The data were expressed as the inverse of the highest dilution of the plasma sample that generated a corrected OD nearest to 0.100, the background. The results for each sample were expressed as fold increase in titer relative to a pretreatment sample. Samples with greater than 4-fold increases were considered to be positive. Samples with circulated levels of MDX-010 may yield lower than actual titers as the anti-MDX-010 antibodies would likely be bound to circulating MDX-010.

CTLA-4 Polymorphism Analysis

Genomic DNA was isolated from peripheral blood mononuclear cells using a QiaAmp kit (Qiagen, Valencia, Calif.). Genotypes for the CTLA-4 JO30 G/A polymorphism were analyzed using PCR-RFLP techniques as previously described (Perrey, C. et al. (1999) Transplant Immunology 7:127-128).

Results

Demographics

Nineteen patients were treated in this phase I trial. Seven patients had resected stage IV, and the remaining twelve had resected stage III disease. Eight patients had received prior immunotherapy with a cell vaccine or alpha-interferon, two had received biochemotherapy, and all patients had undergone surgery. Median time from primary diagnosis was 20 months. Seven patients received MDX-010 at 0.3 mg/kg per dose (cohort 1), seven received 1.0 mg/kg (cohort 2), and five received 3.0 mg/kg (cohort 3). Due to FDA requirements, the first seven patients were dosed every four weeks with MDX-010 only if their serum antibody level was ≦2 ug/ml one week prior to the scheduled infusion. This requirement was lifted after patient number eight in the second cohort.

Autoimmune Toxicity

Toxicities described in prior peptide vaccine trials were also observed in this trial, including local pain, swelling, granuloma formation, with fevers and flu-like symptoms in the majority of patients, principally of grades I and II. The extraordinary toxicities observed were uveitis and gastrointestinal (GI) toxicity in one patient and a rash in two other patients from cohort 2, and GI toxicity in all five patients from cohort 3. Three patients from cohort 3 experienced grade II diarrhea and the remaining two patients from that cohort developed grade III diarrhea. The patient from cohort 2 who developed uveitis and grade III bloody diarrhea required hospitalization. One patient from cohort 3 required admission for severe grade III abdominal pain and grade II diarrhea necessitating intravenous opiates and supportive management. Eight patients had evidence of toxicities that were felt related to MDX-010 and that may have been autoimmune in etiology Patient 8 in cohort 2 developed a grade II rash on her left breast after the second MDX-010 injection with vaccine. A biopsy revealed a deep dermal infiltrate of CD4+ T cells with thickened dermis and peri-vascular cuffing, without true vasculitis. A lesser infiltrate with CD8+ T cells was also seen. The patient's vaccination regimen was not interrupted. The rash resolved slowly over six months.

Patient 19 in cohort 2 developed self-limited grade II diarrhea after the first MDX-010 infusion. One week following his second infusion, the patient presented with bloody diarrhea, visual impairment, photophobia, fevers and fatigue. Colonoscopy revealed diffuse inflammation of the rectum without ulceration. The rectal biopsy obtained at time of colonoscopy revealed a predominant CD4+ T cell infiltrate sparing the glands. A lesser infiltrate with CD8+ T cells was also seen in the rectal interstitium. This was the only patient to require steroids for the treatment of uveitis and colitis, with resolution of all symptoms within 60 days.

Laboratory Values

Total white blood cell count (WBC), hemoglobin and platelet counts were assessed on days 0, 28 and 56 during the first three MDX-010 treatments. There were no significant changes in any hematologic parameter over time or between cohorts.

Flow cytometry analyses were performed on PBMC specimens at the time of the first, second and third MDX-010 infusions as above. CD3+, CD4+, CD8+ T cells, CD56+ NK cells, and CD19+ B cells were enumerated. There were no consistent changes over time in those subsets. Activation markers CD69 and HLA-DR, as well as regulatory marker CD25 were also measured on T cells. The only significant change noted was an increase over time in CD4+/HLA-DR+ T cells at the 1 mg/kg and 3 mg/kg doses, reflective of an increased population of activated T helper cells.

Autoimmune Parameters

Because autoimmune side effects resulting from inhibition of CTLA-4 signaling by MDX-010 had been described in prior trials, serologic and organ function alterations associated with autoimmune reactions were monitored. Erythrocyte sedimentation rate (ESR), anti-nuclear antibodies (ANA), and thyroid stimulating hormone were measured every two months in all patients. No changes in serologic or TSH results were noted. In particular, no changes were noted in ESR or ANA even in patients with significant evidence of skin or GI autoimmune side effects. Patients underwent ophthalmologic evaluation with slit lamp examination every 2 months. In patient 19, who developed uveitis after the second dose of vaccine/MDX-010, clear signs of inflammatory cells in the anterior chamber were seen on slit lamp exam which slowly resolved over 60 days after topical prednisolone eye drops twice a day and tapering intravenous Decadron then oral prednisone over four weeks. That was the only symptomatic abnormality noted on any ophthalmologic examination.

pK Assays

Pharmacokinetic assays for serum MDX-010 levels were performed over six hours at the time of the first infusion. Trough levels were drawn before each subsequent infusion, and peak samples were obtained one hour after each infusion. Dose-dependent peak values, up to 80-90 ug/ml, were noted 240 minutes following the first infusion. Trough values of 10 ug/ml occurred in patients receiving the highest dose of MDX-010. This level is well within the active range of the antibody when used to inhibit CTLA-4 signaling in vitro.

Immune Responses

Eighteen of 19 patients completed leukapheresis prior to initiating the trial, and six months following vaccination with peptides/IFA and MDX-010. Analyses of immune response to MART-1, gp100 and tyrosinase were performed using ELISPOT and tetramer assays.

ELISPOT is a functional assay that measures antigen-specific activation of CD8+ T cells that secrete gamma-interferon. Eighteen patients had samples that were evaluated. Seven (39%) patients had a statistically significant immune response to gp100₂₀₉₋₂₁₇ (210M) following vaccination. Only 3 (17%) patients had a statistically significant increase in reactivity to MART-1₂₆₋₃₅ (27L), although an additional four patients had pre-existing MART-1 immunity. High levels of pre-vaccine reactivity to MART-1 were noted, whereas pre-vaccine reactivity to gp100 was generally low. There was no clear correlation of ELISPOT reactivity to vaccine dose or to the development of autoimmune toxicity, although the limited numbers of patients precludes making definitive correlations. A similar proportion of patients had an immune response to the wild type gp100₂₀₉₋₂₁₇ ( 6/18) and MART-1₂₇₋₃₅ peptides ( 3/18), but with a lower amplitude of response. No significant ELISPOT reactivity to tyrosinase was observed.

The tetramer assay enumerates single cell CD8+ T cell populations that are specific for a MHC-class I peptide combination. The results of gp100₂₀₉₋₂₁₇ (210M) tetramer assays pre- and post-vaccine at month number 6 following six vaccinations determined. No pre-existing reactivity to gp100 was observed, but following vaccination 8/16 (50%) patients had a significant increase in the number of gp100-₂₀₉₋₂₁₇ (210M) specific T cells. Nine of sixteen (56%) patients were noted to have a significant increase in number of MART-1-₂₆₋₃₅ (27L)-specific T cells following vaccination. The strong pre-existing reactivity to MART-1 is demonstrated clearly. No significant tetramer reactivity to tyrosinase was observed. A similar proportion of patients had evidence of reactivity to the gp100₂₀₉₋₂₁₇ ( 7/16) and MART-1₂₇₋₃₅ ( 8/16) wild-type peptides, but a lower level of reactivity was observed.

Flow Cytometry for CCR9 Expression

T cell homing receptors for skin (CLA and CCR4) and gastrointestinal mucosa (CCR9) might be up-regulated in patients treated with MDX-010/vaccine, accounting for cutaneous and GI manifestations observed in patients with autoimmune side effects. Flow cytometry analyses were performed using whole PBMC specimens, obtained prior to and six months after initiation of the regimen, then stained with antibodies for the above markers. Results for CCR9 expression were determined and indicate that there is a trend for increased expression of CCR9 on CD4+ T cells after MDX-01O plus vaccination, especially in the two higher dose cohorts. Eight of thirteen patients analyzed (62%) had significant increases in staining of CD4+ T cells for CCR9. Three of four patients tested in the high dose cohort had increases, compared with only one of five from the lower dose cohorts. No changes were noted for CD4/CLA or CD4/CCR4 staining.

Polymorphisms

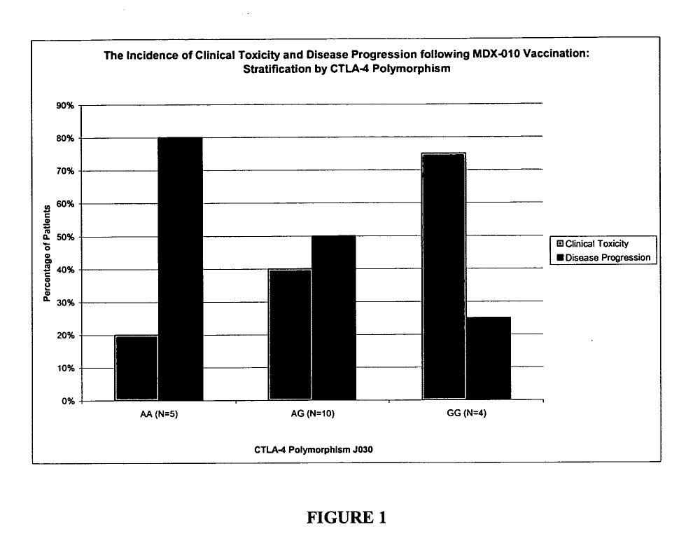

Several single nucleotide polymorphisms for CTLA-4 have been identified. One of these, JO30, encodes three alleles that correlate with the level of expression CTLA-4 expression on T cells. The GG allele for that polymorphism correlated with “low” CTLA-4 levels and was shown to be associated with juvenile onset diabetes and other autoimmune disorders (Ueda et al. (2003) Nature 423:506-511). We hypothesized that patients with “low” CTLA-4 alleles (GG) would have a higher chance of developing autoimmunity with CTLA-4 blockade, and that “high” CTLA-4 alleles (AA or AG) would have a lesser effect from MDX-010 blockade and a lower chance of developing autoimmunity. In fact, 3 of 4 (75%) patients with the “low” CTLA-4 allele (GG) developed autoimmune symptoms and only 1 of these 4 (25%) patients has relapsed. All four patients received the 1 mg/kg or 3 mg/kg dose of MDX-0 10. Of the remaining 15 patients with either the AA or AG alleles, only 5 (33%) developed autoimmune symptoms and 10 (67%) have relapsed. The results are summarized in the graph shown in FIG. 1.

Clinical Results

With 24 months of follow-up, eleven of the nineteen patients treated on this trial have relapsed, and three have died. Of eleven patients with no evidence of autoimmunity, nine (82%) have relapsed and two have died. Median time to relapse was 7 months. Eight of 19 (42%) patients showed signs of autoimmune symptoms; two have relapsed, and one has died. All five (100%) patients in the highest dose cohort (3 mg/kg) had evidence of autoimmunity; two (40%) have relapsed and four are alive, one with disease. Of the 14 patients in the two lower dose cohorts, only 3 (21%) suffered autoimmune effects and nine (64%) patients have relapsed and two have died. These results raise the possibility of a correlation between development of autoimmunity and lack of relapse. Interestingly, no consistent evidence was found of serologic autoimmunity, and no patient developed anti-MDX-010 antibody responses.

### EXAMPLE 2

### Assessment of CTLA-4 Polymorphisms

Susceptibility to autoimmune disorders such as Graves' disease, autoimmune hypothyroidism (AIH) and type 1 diabetes (T1D) has been mapped to a non-coding 6.1 kb 3′ region of CTLA-4 (Ueda, H. et al. (2003) Nature 423:506-511, the entire contents of which, including Supplementary Information A and B are hereby specifically incorporated by reference). In this study, 108 single nucleotide polymorphisms (SNPs) and the CTLA-4 (AT)n −3′ untranslated region (UTR) marker were genotyped in 384 cases of Graves' disease and 652 controls, in 228 cases of AIH and 844 controls and in 3,671 T1D families. The seven SNPs most closely associated with susceptibility to autoimmune disease were the CT60, JO30, JO31, JO27_{—}1, CTAF343, rs1863800 and MH30 markers. The sequences of these SNPs are as follows, wherein the polymorphic nucleotide position is in brackets, with the first nucleotide representing the disease-associated allele and the second nucleotide representing the non-disease associated allele:

| | |
|---|---|
| CT60 | |
| TTTTGATTTCTTCACCACTATTTGGGATATAAC[G/A]TGGGTTAACACAGACATA | (SEQ ID NO:1) |
| JO30 | |
| CGGACCTCTTGAGGTCAGGAGTTC[G/A]AGACCAGCCTGGCCAACATGGTGA | (SEQ ID NO:2) |
| JO31 | |
| AACAGTCTGTCAGCAAAGCC[G/T]GCAGTACACTGAGAAAGCTCCTATT | (SEQ ID NO:3) |
| JO27 1 | |
| CCAGAAGTTGAAGTGTAGGAA[T/C]ATCTGGGGTCAAAGCAAAAAAAGACTTT | (SEQ ID NO:4) |
| CTAF343 | |
| TGGGAGTATTTTACTGTGCTAAAA[T/C]ACATTTAGCATGGGCTGTTATATCTTATGAC | (SEQ ID NO:5) |
| Rs1863800 | |
| GATAAAAAGGAACTGTTTAAA[C/T]TGATAGTAAAGAAAAGCCTTAAATTTTTGG | (SEQ ID NO:6) |
| MH30 | |
| AATAAAAACAGAATAAAACAAT[G/C]AGAAAATTTTCACCTTTATTTAATTAGCAGA | (SEQ ID NO:7) |

To assess CTLA-4 polymorphism expression in a subject who is to undergo CTLA-4 blocking agent therapy, genomic DNA is isolated from peripheral blood lymphocytes of the subject by standard methods. A preferred method for evaluating SNPs is the Invader® technology described in Mein, C. et al. (2000) Genome Research 10:330-343 (the contents of which is expressly incorporated herein by reference). Alternatively, SNPs can be evaluated using an amplification refractory mutation system-polymerase chain reaction (ARMS-PCR) technology such as described in Perrey, C. et al. (1999) Transplant Immunol. 7:127-128. Probe sets for specifically detecting the polymorphism(s) to be analyzed are designed and synthesized according to known methodologies.

1. A method for predicting responsiveness of a subject to therapy with a CTLA-4 blocking agent, comprising:
assaying for at least one CTLA-4 polymorphism in the subject; and
predicting responsiveness of the subject to therapy with a CTLA-4 blocking agent based on presence or absence of a CTLA-4 polymorphic allele in the subject.

2. The method of claim 1, which further comprises selecting a treatment regimen with a CTLA-4 blocking agent based upon presence or absence of the CTLA-4 polymorphic allele in the subject.

3. The method of claim 2, which further comprises administering the CTLA-4 blocking agent to the subject according to the treatment regimen such that responsiveness to antigenic stimulation is increased in the subject.

4. The method of claim 1, wherein the CLTA-4 polymorphism is a single nucleotide polymorphism (SNP) associated with susceptibility to autoimmune disease.

5. The method of claim 4, wherein the SNP is a JO30 G/A polymorphism.

6. The method of claim 4, wherein the SNP is selected from the group consisting of a JO30 G/A polymorphism, a CT60 G/A polymorphism, a JO31 G/T polymorphism, a JO27_{—}1 T/C polymorphism, a CTAF343 T/C polymorphism, a rs1863800 C/T polymorphism and a MH30 G/C polymorphism.

7. The method of claim 1, wherein the CTLA-4 polymorphism is a polymorphism associated with susceptibility to autoimmune disease selected from the group consisting of a CTLA-4 exon I position 49 A/G polymorphism, a CTLA-4 promoter position −318 C/T polymorphism, a CTLA-4 intron 1 position 1822 C/T polymorphism and a CTLA-4 exon 3 dinucleotide (AT)n repeat polymorphism.

8. The method of claim 1, wherein a CTLA-4 polymorphic allele associated with increased susceptibility to autoimmune disease is present in the subject and the subject is predicted to have increased responsiveness to therapy with a CTLA-4 blocking agent as compared to a subject not carrying the allele.

9. The method of claim 8, wherein increased responsiveness to therapy with a CTLA-4 blocking agent includes at least one response selected from the group consisting of: increased T cell responsiveness to antigenic stimulation, increased anti-tumor activity, increased autoimmune breakthrough events, increased clinically adverse events and increased serological adverse events.

10. The method of claim 1, wherein a CTLA-4 polymorphic allele associated with decreased susceptibility to autoimmune disease is present in the subject and the subject is predicted to have decreased responsiveness to therapy with a CTLA-4 blocking agent as compared to a subject not carrying the allele.

11. The method of claim 10, wherein decreased responsiveness to therapy with a CTLA-4 blocking agent includes at least one response selected from the group consisting of: decreased T cell responsiveness to antigenic stimulation, decreased anti-tumor activity, decreased autoimmune breakthrough events, decreased clinically adverse events and decreased serological adverse events.

12. The method of claim 2, wherein the subject expresses two G alleles (G/G genotype) at a JO30 G/A polymorphism and the treatment regimen that is selected is a reduced treatment regimen as compared to a standard treatment regimen.

13. The method of claim 2, wherein the subject expresses a genotype selected from the group consisting of two G alleles (G/G genotype) at a JO30 G/A polymorphism, two G alleles (G/G genotype) at a CT60 G/A polymorphism, two G alleles (G/G genotype) at a JO31 G/T polymorphism, two T alleles (T/T genotype) at a JO27_{—1} T/C polymorphism, two T alleles (T/T genotype) at a CTAF343 T/C polymorphism, two C alleles (C/C genotype) at a rs1863800 C/T polymorphism and two G alleles (G/G genotype) at a MH30 G/C polymorphism and the treatment regimen that is selected is a reduced treatment regimen as compared to a standard treatment regimen.

14. The method of claim 12, wherein the reduced treatment regimen comprises use of a lower dose of CTLA-4 blocking agent, less frequent administration of the CTLA-4 blocking agent or shorter treatment duration with the CTLA-4 blocking agent as compared to a standard treatment regimen.

15. The method of claim 2, wherein the subject expresses two A alleles (A/A genotype) at a JO30 G/A polymorphism and the treatment regimen that is selected is an increased treatment regimen as compared to a standard treatment regimen.

16. The method of claim 2, wherein the subject expresses a genotype selected from the group consisting of two A alleles (A/A genotype) at a JO30 G/A polymorphism, two A alleles (A/A genotype) at a CT60 G/A polymorphism, two T alleles (T/T genotype) at a JO31 G/T polymorphism, two C alleles (C/C genotype) at a JO27_{—}1 T/C polymorphism, two C alleles (C/C genotype) at a CTAF343 T/C polymorphism, two T alleles (T/T genotype) at a rs1863800 C/T polymorphism and two C alleles (C/C genotype) at a MH30 G/C polymorphism, and the treatment regimen that is selected is an increased treatment regimen as compared to a standard treatment regimen.

17. The method of claim 15, wherein the increased treatment regimen comprises use of a higher dose of CTLA-4 blocking agent, more frequent administration of the CTLA-4 blocking agent or longer treatment duration with the CTLA-4 blocking agent as compared to a standard treatment regimen.

18. (canceled)

19. The method of claim 1, wherein the CTLA-4 blocking agent is an anti-CTLA-4 monoclonal antibody.

20. The method of claim 19, wherein the anti-CTLA-4 monoclonal antibody is a human monoclonal antibody.

21. The method of claim 1, wherein the subject is a human.

22-31. (canceled)

32. A method for predicting an increased susceptibility to an autoimmune disorder of a subject undergoing therapy with a CTLA-4 blocking agent, comprising:
(a) assaying for at least one CTLA-4 polymorphism in the subject undergoing therapy with a CTLA-4 blocking agent; and
(b) predicting increased susceptibility to an autoimmune disorder of the subject based on the presence of a CTLA-4 polymorphic allele in the subject.

33. The method of claim 32, wherein the CTLA-4 polymorphic allele is a two G CTLA-4 polymorphic allele (G/G genotype) selected from the group consisting of a CT60 G/A polymorphism, a JO30 G/A polymorphism, a JO31 G/T polymorphism, a JO27_{—}1T/C polymorphism, a CTAF343 T/C polymorphism, a rs1863800 C/T polymorphism and a MH30 G/C polymorphism.

34. The method of claim 32, which comprises reducing a CTLA-4 blocking agent treatment regimen compared to a standard treatment regimen in a subject predicted to have increased susceptibility to an autoimmune disorder.

35. A method for predicting decreased susceptibility to an autoimmune disorder of a subject undergoing therapy with a CTLA-4 blocking agent, comprising:
(a) assaying for at least one CTLA-4 polymorphism in the subject undergoing therapy with a CTLA-4 blocking agent; and
(b) predicting decreased susceptibility to an autoimmune disorder of the subject based on the presence of a CTLA-4 polymorphic allele in the subject.

36. The method of claim 35, wherein the CTLA-4 polymorphic allele is a genotype selected from the group consisting of two A alleles (A/A genotype) at a CT60 G/A polymorphism, two T alleles (T/T genotype) at a JO31 G/T polymorphism, two C alleles (C/C genotype) at a JO27_{—}1 T/C polymorphism, two C alleles (C/C genotype) at a CTAF343 T/C polymorphism, two T alleles (T/T genotype) at a rs1863800 C/T polymorphism and two C alleles (C/C gentoype) at a MH30 G/C polymorphism.

37. The method of claim 35, which comprises increasing a CTLA-4 blocking agent treatment regimen compared to a standard treatment regimen in a subject predicted to have decreased susceptibility to an autoimmune disorder.

38. The method of claim 32, wherein the CTLA-4 blocking agent is an anti-CTLA-4 antibody.

39. The method of claim 38, wherein the anti-CTLA-4 antibody is MDX-010.
